(19) United States
(12) Patent Application Publication
(10) Pub. No.: US 2014/0288446 A1
(43) Pub. Date: Sep. 25, 2014

(54) Apparatus for Monitoring Physiological Condition

(71) Applicant: AVITA CORPORATION, New Taipei City (TW)

(72) Inventor: Chun-Ho Lee, New Taipei City (TW)

(73) Assignee: AVITA CORPORATION, New Taipei City (TW)

(21) Appl. No.: 14/220,098

(22) Filed: Mar. 19, 2014

(30) Foreign Application Priority Data
Mar. 19, 2013 (TW) 102205060

(51) Int. Cl. A61B 5/022; A61B 5/026

(52) U.S. Cl. CPC A61B 5/022; A61B 5/026
USPC 600/493

(57) ABSTRACT

An apparatus for monitoring a physiological condition includes a signal-acquiring unit, an inflating-deflating unit, and a central processing system electrically coupled to the signal-acquiring unit and the inflating-deflating unit. The signal-acquiring unit is used for acquiring a first standard pulse signal and a first reactive hyperemia pulse signal at a specific part of a living being. The inflating-deflating unit is used for selectively inflating and deflating the specific part. The central processing system is capable of transforming the first standard pulse signal to a second standard pulse signal and transforming the first reactive hyperemia pulse signal to a second reactive hyperemia pulse signal using a nonstationary and nonlinear transfer function, respectively, to determine an endothelial function coefficient according to the second standard pulse signal and the second reactive hyperemia pulse signal, thus to analyze a physiological condition of the living being.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### CROSS REFERENCE OF RELATED APPLICATION

This Non-provisional application claims priority under 35 U.S.C. §119(a) on Patent Application No(s). 102205060 filed in Taiwan, Republic of China on Mar. 19, 2013, the entire contents of which are hereby incorporated by reference.

### BACKGROUND OF THE PRESENT INVENTION

1. Field of Invention

This invention relates to an apparatus for monitoring a physiological condition and, more particularly, to an apparatus for monitoring a physiological condition by transforming measured blood pressure signals using a nonstationary and nonlinear transfer function.

2. Description of Related Arts

Cardiovascular diseases account for 30 percent of the top ten causes of death, thus becoming the leading killer of human being. Risk factors for the cardiovascular diseases are diabetes, hypertension, hyperlipidemia, smoking and so on. In addition, many research reports indicate that erectile dysfunction (ED) happens, often sooner than the cardiovascular diseases. However, cultural backgrounds and traditional concepts may make male patients shy away from getting medical attention, thus losing opportunities of early diagnosis and prevention of the cardiovascular diseases. According to the National Institutes of Health, the ED occurs when a man can no longer get or keep an erection firm enough for sexual intercourse.

Both the ED and the cardiovascular diseases belong to vascular diseases resulting from vascular endothelial dysfunction, and therefore an assessment on endothelial function can be regarded as a leading indicator. That is, endothelial dysfunction can provide an early warning of the vascular diseases. Besides, according to recent research reports, autonomic nervous dysfunction may be another sign of diseases. Both of them play a significant role in physiological systems. However, to clarify weightings of factors, a further research and discussion should be needed.

Simply put, vascular endothelial function may directly affect the degree of blood vessel dilatation, and therefore the vascular endothelial function can be indirectly reflected by measuring of the degree of blood vessel dilatation.

At present, standard methods for assessing the vascular endothelial function are still to use ultrasound or Endo-PAT 2000 apparatuses, while an assessment of autonomic nervous function should rely on heart rate variability (HRV), baroreflex sensitivity (BRS), or muscle sympathetic nervous activity apparatuses. Although the vascular endothelial dysfunction has been regarded as an early sign of the vascular diseases, the two standard apparatuses are so expensive and inconvenient to use, and therefore they are only used in academic research. Accordingly, to assess the endothelial function and the autonomic nervous function, hospitals have to use different apparatuses, respectively, at present. It is quite inconvenient for subjects. If measuring apparatuses and assessment indicators suitable for home measurement are developed, the subjects are more willing to be measured, thus achieving the effect of prevention better than cure.

### SUMMARY OF THE PRESENT INVENTION

One objective of this invention is to provide an apparatus for monitoring a physiological condition to achieve home measurement.

To achieve the above objective, the invention adopts the following technology means.

According to one aspect of the invention, the invention provides an apparatus for monitoring a physiological condition including a signal-acquiring unit, an inflating-deflating unit, and a central processing system. The signal-acquiring unit is used for acquiring a first standard pulse signal and a first reactive hyperemia pulse signal at a specific part of a living being. The inflating-deflating unit is used for selectively inflating and deflating the specific part of the living being. The central processing system is electrically coupled to the signal-acquiring unit and the inflating-deflating unit. The central processing system is capable of transforming the first standard pulse signal to a second standard pulse signal and transforming the first reactive hyperemia pulse signal to a second reactive hyperemia pulse signal using a nonstationary and nonlinear transfer function, respectively, to determine an endothelial function coefficient of the living being according to the second standard pulse signal and the second reactive hyperemia pulse signal, thus to analyze a physiological condition of the living being.

These and other features, aspects, and advantages of the present invention will become better understood with regard to the following description, appended claims, and accompanying drawings.

### BRIEF DESCRIPTION OF THE DRAWINGS

FIG. 1 is a block diagram of an apparatus for monitoring a physiological condition according to one embodiment of the invention;

FIG. 2 is a schematic diagram of amplitude variation of arm pulse signals before and after reactive hyperemia according to one embodiment of the invention;

FIG. 3 is a schematic diagram of a component of a variation trend of a blood vessel dilatation according to one embodiment of the invention;

FIG. 4 is a schematic diagram of computing time intervals of arm blood pressure pulse signals according to one embodiment of the invention;

FIG. 5 is a schematic diagram of a series of successive time intervals of arm pulse signals according to one embodiment of the invention;

FIG. 6 is a schematic diagram of a variation of an energy spectrum according to one embodiment of the invention; and

FIG. 7 is a flow chart of a method for monitoring a physiological condition according to one embodiment of the invention.

### DETAILED DESCRIPTION OF THE PREFERRED EMBODIMENT

At present, physiological abnormalities include ED, sleep apnea, hypertension, or arteriosclerosis and so on. Most of these belong to cardiovascular diseases. Early prevention should be conducted in various directions, and thus preventive medicine can be really practiced in the grass roots. An apparatus provided by the invention can help to measure vascular endothelial function and autonomic nervous function. Further, it can be used akin to using a sphygmomanometer and has advantages of a small size and a low cost. Accordingly, it is suitable for home measurement.

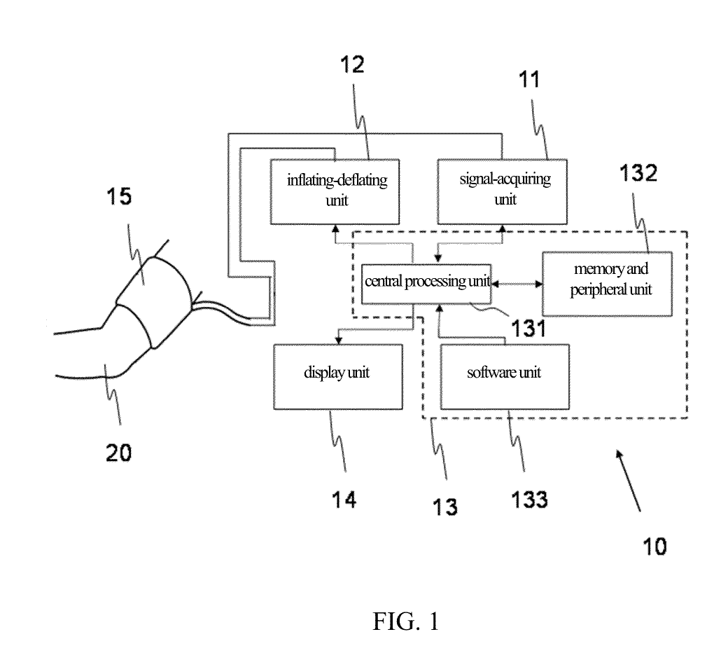

FIG. 1 is a block diagram of an apparatus for monitoring a physiological condition according to one embodiment of the invention. In FIG. 1, the apparatus for monitoring a physiological condition 10 mainly includes a signal-acquiring unit 11, an inflating-deflating unit 12, a central processing system 13, and a display unit 14. The signal-acquiring unit 11 contacts a specific part of a living being 20 directly or indirectly and is used for acquiring a first standard pulse signal and a first reactive hyperemia pulse signal at the specific part of the living being 20. The inflating-deflating unit 12 contacts the specific part of the living being 20 directly or indirectly and is used for selectively inflating and deflating the specific part of the living being 20. The signal-acquiring unit 11 and the inflating-deflating unit 12 can be used akin to using a common electronic sphygmomanometer. That is, when a user (living being 20) uses the inflating-deflating unit 12 to inflate an arm to a baseline pressure (40±3 mmHg), the signal-acquiring unit 11 acquires the first standard pulse signal. Afterwards, the inflating-deflating unit 12 inflates the arm to an occlusion pressure which is the sum of the baseline pressure and a systolic blood pressure of the user (living being 20). When the inflating-deflating unit 12 deflates the arm to the baseline pressure, the signal-acquiring unit 11 acquires the first reactive hyperemia pulse signal.

The central processing system 13 is electrically coupled to the signal-acquiring unit 11 and the inflating-deflating unit 12. The central processing system 13 includes a central processing unit 131, a memory and peripheral unit 132, and a software unit 133, and it transforms the first standard pulse signal to a second standard pulse signal and transforms the first reactive hyperemia pulse signal to a second reactive hyperemia pulse signal using a nonstationary and nonlinear transfer function, respectively, to determine an endothelial function coefficient of the user (living being 20) according to the second standard pulse signal and the second reactive hyperemia pulse signal, thus to analyze and display a physiological condition of the user (living being 20).

In addition, the apparatus for monitoring a physiological condition 10 further includes a cuff 15 placed around the specific part (arm) of the living being 20. The signal-acquiring unit 11 and the inflating-deflating unit 12 are disposed at the cuff 15 to acquire the first standard pulse signal and the first reactive hyperemia pulse signal at the specific part (arm) of the living being 20 and to selectively inflate and deflate the specific part (arm) of the living being 20.

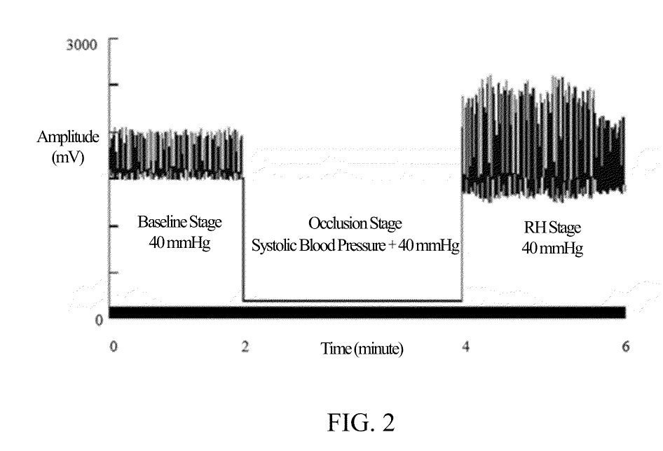

FIG. 2 is a schematic diagram of amplitude variation of arm pulse signals before and after reactive hyperemia according to one embodiment of the invention. In FIG. 2, indicators are quantified mainly from static and dynamic views in analyzing vascular endothelial function (endothelial function coefficient). As for the static indicator, a dilatation index (DI) is defined according to a flow-mediated dilation (FMD) theory. The apparatus for monitoring a physiological condition in the embodiment can acquire the successive variation of the pulse signal before and after inflating the arm. The average pulse amplitude in one minute starting from the second minute in a reactive hyperemia (RH) stage is divided by the average pulse amplitude in a baseline stage, and then the natural logarithm of the obtained value is calculated thus to obtain the DI. The greater the DI, the better the endothelial function.

When reactive hyperemia happens after deflating the arm from the occlusion pressure, endothelial cells may generate and release nitric oxide (NO), thus allowing the blood vessel to dilate. Human being is factually a dynamic and complex system. Accordingly, although the static indicator has been proved to have good sensitivity and accuracy in clinical research, it may miss a lot of underlying or subtle physiological phenomena if only the static indicator is used to quantify the endothelial function. For example, when the endothelial cells are stimulated in response to the occlusion pressure, the reaction speed and the maintaining time for the blood vessel to dilate to the maximum degree may differ between different subjects since each subject releases different amounts of nitric oxide.

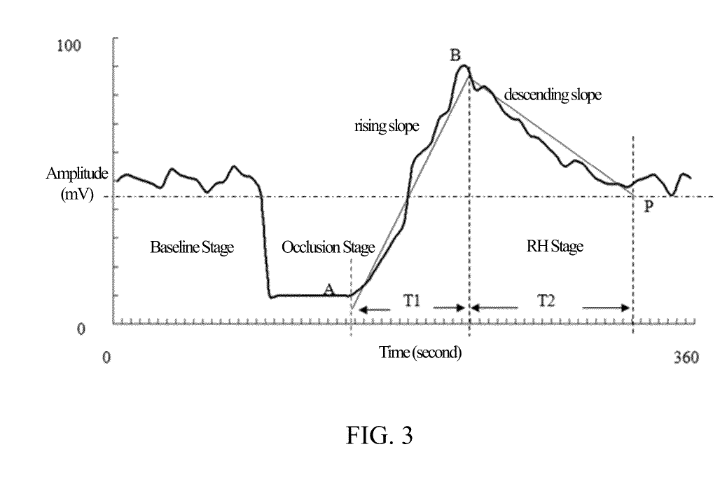

FIG. 3 is a schematic diagram of a component of a variation trend of a blood vessel dilatation according to one embodiment of the invention. Here the indicators are quantified from the dynamic view. In FIG. 3, a nonstationary and nonlinear transfer function is used to dynamically assess the endothelial function in the embodiment. The nonstationary and nonlinear transfer function may be Hilbert-Huang transformation (HHT) algorithm. Especially, empirical mode decomposition (EMD) of the HHT algorithm is used to transform the first standard pulse signal to a second standard pulse signal and to transform the first reactive hyperemia pulse signal to a second reactive hyperemia pulse signal, and then the endothelial function is analyzed dynamically. The EMD regards the instantaneous variation scale of the signal as the energy and decomposes it directly. In detail, the original signal is decomposed into a plurality of intrinsic mode functions (IMF), and each IMF is regarded as a basis of the original signal. Accordingly, the analyzed signal can be nonlinear or nonstationary, and thus the bases can completely show physical characteristics of the original signal. The wave amplitude in the baseline stage before the occlusion stage (i.e. the second standard pulse signal) is averaged as a horizontal threshold of the whole variation trend, and the time (second) and the amplitude (millivolt) at the locations A, B, and P of the second reactive hyperemia pulse signal are calculated, respectively, using the threshold line. Further, a rising slope and a time difference (T1) from the location A to the location B (first section) are calculated, and a descending slope and a time difference (T2) from the location B to the location P (second section) are calculated. The rising slope is defined as the rising speed and time from the endothelial cells releasing nitric oxide to the blood vessel dilating to the maximum degree, and the descending slope is defined as the recovery rate and time from the blood vessel dilating to the maximum degree to the blood vessel recovered to a normal state. Dynamic variation of the blood vessel dilatation after the vascular endothelial cells release nitric oxide can be assessed by calculating the rising slope and the descending slope. Such dynamic indicators can be used to assess the physiological condition of the living being such as the time from relaxation to complete erection of the penis and the maintaining time of the erection. Afterwards, the variation degree of the endothelial function can be completely presented by quantifying the indicators from the static and dynamic views. Accordingly, the endothelial function coefficient can be determined according to the rising slope and the descending slope.

The invention has proposed an innovative and different method for assessing the endothelial function and quantifying the indicators. However, if the indicator of the autonomic nervous function can be further quantified, assessment and prevention of the ED and the cardiovascular diseases can achieve the optimal effect. In the invention, the autonomic nervous function is assessed using a nonstationary and nonlinear transfer function which may be Hilbert-Huang transformation (HHT) algorithm, especially empirical mode decomposition (EMD) and Hilbert transformation (HT) of the HHT algorithm. In detail, a standard autonomic nerve parameter can be obtained according to the first standard pulse signal, and a reactive hyperemia autonomic nerve parameter can be obtained according to the first reactive hyperemia pulse signal. Further, the autonomic nervous function of the living being can be determined according to the standard autonomic nerve parameter and the reactive hyperemia autonomic nerve parameter.

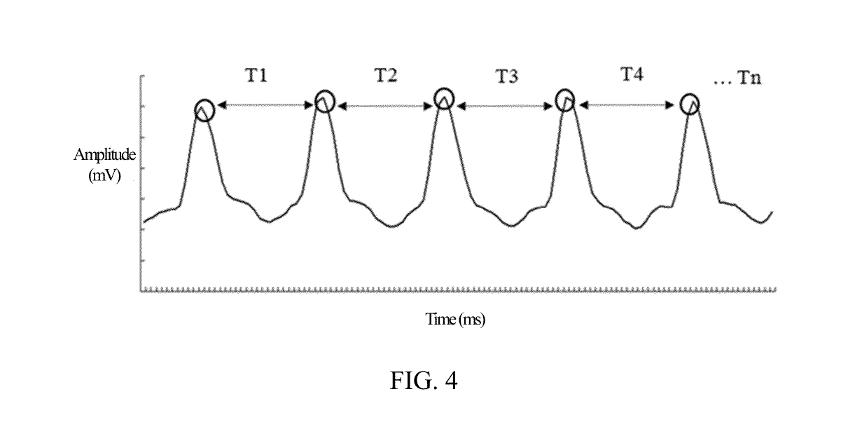

FIG. 4 is a schematic diagram of computing time intervals of arm blood pressure pulse signals according to one embodiment of the invention. In FIG. 4, to assess the autonomic nervous function in the embodiment, each of the blood pressure pulse signals in the baseline stage and the RH stage in FIG. 2 is recorded, and the difference of peak time between two adjacent pulse signals is calculated and is expressed as the time series T={T1, T2, T3, T4, . . . , Tn} for time-frequency analysis.

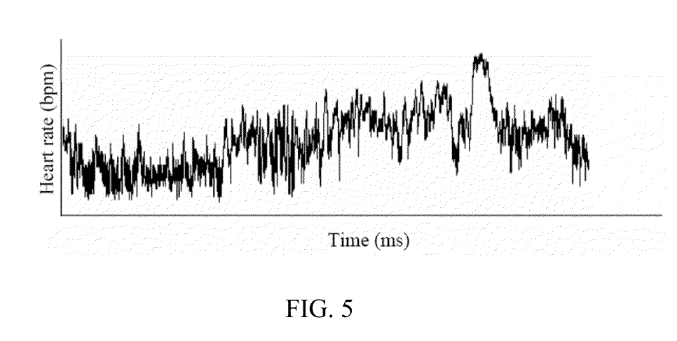

FIG. 5 is a schematic diagram of a series of successive time intervals of arm pulse signals according to one embodiment of the invention. In FIG. 5, the nonstationary characteristic of the signal may increase the degree of irregularity of the time series thus to affect the accuracy of spectral analysis, and therefore during spectral transformation, trend is first removed from the time series using the EMD of the HHT algorithm to obtain an accurate result of the spectral analysis.

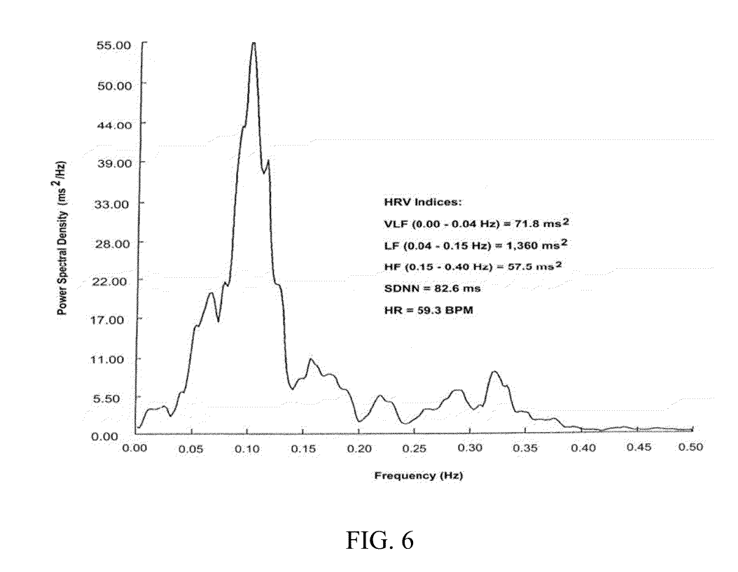

FIG. 6 is a schematic diagram of a variation of an energy spectrum according to one embodiment of the invention. In FIG. 6, after the signal decomposed using the EMD is transformed using the HT, energy variation of each band of frequencies can be obtained. For example, high frequency power (HF) is ranged from 0.15 to 0.4 Hz indicating variance of normal to normal interval in the high frequency band and representing an indicator of parasympathetic nervous activity; low frequency power (LF) is ranged from 0.04 to 0.15 Hz indicating variance of normal to normal interval in the low frequency band and representing an indicator of sympathetic nervous activity or interaction between sympathetic nerve and the parasympathetic nerve; very low frequency power (VLF) is ranged from 0.003 to 0.04 Hz indicating variance of normal to normal interval in the very low frequency band. Accordingly, the balance states of sympathetic nervous activity and the parasympathetic nervous activity before and after occlusion pressure can be observed.

The vascular endothelial function and the autonomic nervous function are coordinated in the operation of the physiological system, and therefore both the vascular endothelial dysfunction and the autonomic nervous dysfunction have been regarded as early signs of the ED and the vascular diseases. The invention provides an apparatus for measuring the vascular endothelial function and the autonomic nervous function at any time, and it is believed that the risk of disease occurrence in the future may be lowered greatly.

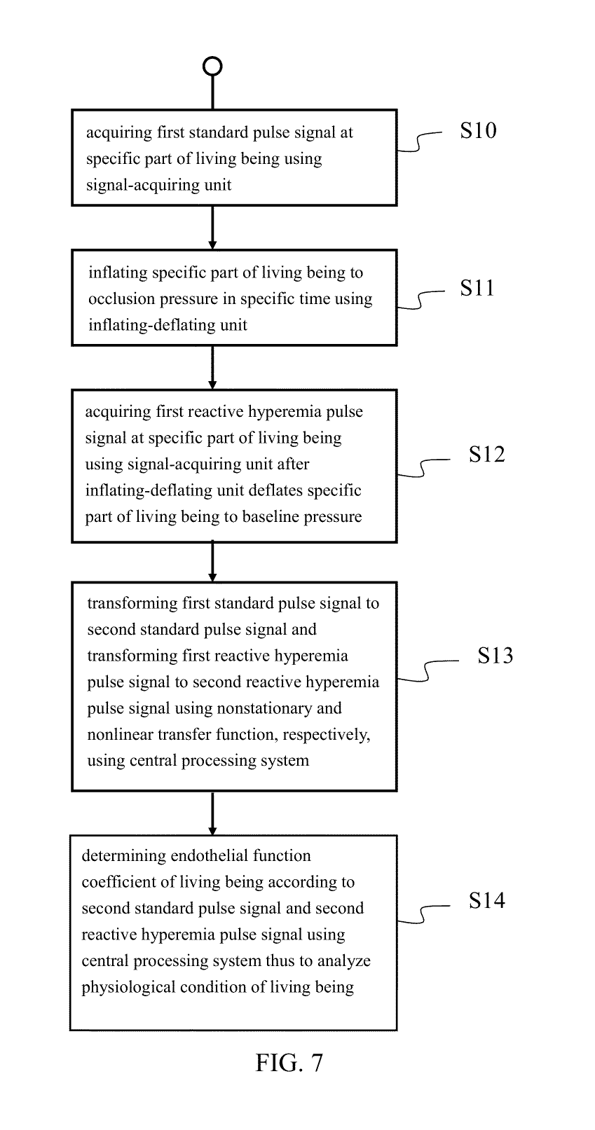

FIG. 7 is a flow chart of a method for monitoring a physiological condition according to one embodiment of the invention. In FIG. 7, the method for monitoring a physiological condition in the embodiment includes the following steps. First, a first standard pulse signal at a specific part of a living being 20 is acquired using a signal-acquiring unit 11 (step S10). The first standard pulse signal may be acquired using the signal-acquiring unit 11 when an inflating-deflating unit 12 inflates the specific part of the living being 20 to a baseline pressure. Second, the specific part of the living being 20 is inflated to an occlusion pressure in a specific time (about 2 minutes) using the inflating-deflating unit 12 (step S11). The occlusion pressure may be the sum of the baseline pressure and a systolic blood pressure of the living being 20. A first reactive hyperemia pulse signal at the specific part of the living being 20 is acquired using the signal-acquiring unit 11 after the inflating-deflating unit 12 deflates the specific part of the living being 20 to the baseline pressure (step S12). The first reactive hyperemia pulse signal may be acquired using the signal-acquiring unit 11 when the inflating-deflating unit 12 deflates the specific part of the living being 20 to the baseline pressure. The first standard pulse signal is transformed to a second standard pulse signal and the first reactive hyperemia pulse signal is transformed to a second reactive hyperemia pulse signal using a nonstationary and nonlinear transfer function, respectively, using a central processing system 13 (step S13). The nonstationary and nonlinear transfer function may be HHT algorithm, especially EMD of the HHT algorithm. Finally, an endothelial function coefficient of the living being 20 is determined according to the second standard pulse signal and the second reactive hyperemia pulse signal using the central processing system 13 thus to analyze a physiological condition of the living being 20 (step S14).

Further, the signal-acquiring unit 11 and the inflating-deflating unit 12 may be disposed at a cuff 15, and the cuff 15 is used for being placed around the specific part of the living being 20.

In addition, in the method for monitoring a physiological condition, the second reactive hyperemia pulse signal can be further divided into a first section and a second section. A rising slope of the first section and a descending slope of the second section are obtained, respectively. The rising slope and the descending slope are used for determining the endothelial function coefficient. The rising slope is defined as the rising speed and time from the endothelial cells releasing nitric oxide to the blood vessel dilating to the maximum degree, and the descending slope is defined as the recovery rate and time from the blood vessel dilating to the maximum degree to the blood vessel recovered to a normal state.

The method for monitoring a physiological condition further includes the steps of obtaining a standard autonomic nerve parameter according to the first standard pulse signal and obtaining a reactive hyperemia autonomic nerve parameter according to the first reactive hyperemia pulse signal using the nonstationary and nonlinear transfer function, respectively, thus to determine an autonomic nervous function of the living being according to the standard autonomic nerve parameter and the reactive hyperemia autonomic nerve parameter. The nonstationary and nonlinear transfer function may be the HHT algorithm, especially the EMD and the HT of the HHT algorithm.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. An apparatus for monitoring a physiological condition, comprising:
a signal-acquiring unit for acquiring a first standard pulse signal and a first reactive hyperemia pulse signal at a specific part of a living being;
an inflating-deflating unit for selectively inflating and deflating the specific part of the living being; and
a central processing system electrically coupled to the signal-acquiring unit and the inflating-deflating unit, the central processing system being capable of transforming the first standard pulse signal to a second standard pulse signal and transforming the first reactive hyperemia pulse signal to a second reactive hyperemia pulse signal using a nonstationary and nonlinear transfer function, respectively, to determine an endothelial function coefficient of the living being according to the second standard pulse signal and the second reactive hyperemia pulse signal, thus to analyze a physiological condition of the living being.

2. The apparatus for monitoring a physiological condition according to claim 1, wherein when the inflating-deflating unit inflates the specific part of the living being to a baseline pressure, the signal-acquiring unit acquires the first standard pulse signal.

3. The apparatus for monitoring a physiological condition according to claim 2, wherein the inflating-deflating unit is capable of inflating the specific part of the living being to an occlusion pressure, and the occlusion pressure is the sum of the baseline pressure and a systolic blood pressure of the living being.

4. The apparatus for monitoring a physiological condition according to claim 3, wherein when the inflating-deflating unit deflates the specific part of the living being to the baseline pressure, the signal-acquiring unit acquires the first reactive hyperemia pulse signal.

5. The apparatus for monitoring a physiological condition according to claim 1, further comprising a cuff placed around the specific part of the living being, wherein the signal-acquiring unit and the inflating-deflating unit are disposed at the cuff.

6. The apparatus for monitoring a physiological condition according to claim 1, wherein the central processing system further divides the second reactive hyperemia pulse signal into a first section and a second section and obtains a rising slope of the first section and a descending slope of the second section, respectively, and the rising slope and the descending slope are used for determining the endothelial function coefficient.

7. The apparatus for monitoring a physiological condition according to claim 1, wherein the central processing system further obtains a standard autonomic nerve parameter according to the first standard pulse signal and obtains a reactive hyperemia autonomic nerve parameter according to the first reactive hyperemia pulse signal using the nonstationary and nonlinear transfer function, respectively, thus to determine an autonomic nervous function of the living being according to the standard autonomic nerve parameter and the reactive hyperemia autonomic nerve parameter.

8. The apparatus for monitoring a physiological condition according to claim 1, wherein the physiological condition comprises erectile dysfunction (ED), sleep apnea, hypertension, or arteriosclerosis.

9. The apparatus for monitoring a physiological condition according to claims 1, wherein the nonstationary and nonlinear transfer function is Hilbert-Huang transformation (HHT) algorithm.

10. A method for monitoring a physiological condition, comprising the steps of:
acquiring a first standard pulse signal at a specific part of a living being using a signal-acquiring unit;
inflating the specific part of the living being to an occlusion pressure in a specific time using an inflating-deflating unit;
acquiring a first reactive hyperemia pulse signal at the specific part of the living being using the signal-acquiring unit after the inflating-deflating unit deflates the specific part of the living being;
transforming the first standard pulse signal to a second standard pulse signal and transforming the first reactive hyperemia pulse signal to a second reactive hyperemia pulse signal using a nonstationary and nonlinear transfer function, respectively, using a central processing system; and
determining an endothelial function coefficient according to the second standard pulse signal and the second reactive hyperemia pulse signal using the central processing system thus to analyze a physiological condition of the living being.

11. The method for monitoring a physiological condition according to claim 10, wherein the first standard pulse signal is acquired using the signal-acquiring unit when the inflating-deflating unit inflates the specific part of the living being to a baseline pressure.

12. The method for monitoring a physiological condition according to claim 10, wherein the occlusion pressure is the sum of a baseline pressure and a systolic blood pressure of the living being.

13. The method for monitoring a physiological condition according to claim 10, wherein the first reactive hyperemia pulse signal is acquired using the signal-acquiring unit when the inflating-deflating unit deflates the specific part of the living being to a baseline pressure.

14. The method for monitoring a physiological condition according to claim 10, wherein the signal-acquiring unit and the inflating-deflating unit are disposed at a cuff, and the cuff is used for being placed around the specific part of the living being.

15. The method for monitoring a physiological condition according to claim 10, wherein the second reactive hyperemia pulse signal is capable of being divided into a first section and a second section, a rising slope of the first section and a descending slope of the second section are obtained, respectively, and the rising slope and the descending slope are used for determining the endothelial function coefficient.

16. The method for monitoring a physiological condition according to claim 10, further comprising the steps of obtaining a standard autonomic nerve parameter according to the first standard pulse signal and obtaining a reactive hyperemia autonomic nerve parameter according to the first reactive hyperemia pulse signal, thus to determine an autonomic nervous function of the living being according to the standard autonomic nerve parameter and the reactive hyperemia autonomic nerve parameter.

17. The method for monitoring a physiological condition according to claim 16, further comprising the steps of obtaining the standard autonomic nerve parameter according to the first standard pulse signal and obtaining the reactive hyperemia autonomic nerve parameter according to the first reactive hyperemia pulse signal using the nonstationary and nonlinear transfer function, respectively.

18. The method for monitoring a physiological condition according to claim 10, wherein the physiological condition comprises ED, sleep apnea, hypertension, or arteriosclerosis.

19. The method for monitoring a physiological condition according to claims 10, wherein the nonstationary and nonlinear transfer function comprises HHT algorithm.

20. The method for monitoring a physiological condition according to claims 17, wherein the nonstationary and nonlinear transfer function comprises HHT algorithm.
